(19) United States
(12) Patent Application Publication
(10) Pub. No.: US 2021/0330940 A1
(43) Pub. Date: Oct. 28, 2021

(54) Extensible Guide for a Tube

(71) Applicant: Anne Margaret Le Clezio, Atholl (ZA)

(72) Inventor: Louis Jean Christian Le Clezio, Atholl (ZA)

(21) Appl. No.: 17/284,134

(22) PCT Filed: Mar. 20, 2019
(86) PCT No.: PCT/ZA2019/050012

§ 371 Date: Apr. 9, 2021

(30) Foreign Application Priority Data
Oct. 16, 2018 (ZA) 2018/06878

(51) Int. Cl. A61M 25/02; A61M 25/01

(52) U.S. Cl. CPC A61M 25/02; A61M 2205/0216; A61M 2025/024; A61M 25/0138

(57) ABSTRACT

A extensible guide that includes a body (10) and means for securing a tube (12) to the body. The body is in the shape of a helix with a substantially constant radius of curvature along the length of the body. The body is axially resiliently deformable between: (i) a neutral condition, in which the body is not subject to axial compressive or tensile forces; (ii) an extended condition, in which the body is subject to an axial tensile force; and (iii) a retracted condition, in which the body is subject to an axial compressive force. When the body is in the neutral condition, the extended condition or the retracted condition, the radius of curvature of the body remains substantially constant along the length of the body. In use, the extensible guide causes the flexible tube secured thereto to assume the helical shape of the body

### BACKGROUND

The present invention relates to an extensible guide. More particularly, the present invention relates to a helical extensible guide for a tube.

Various extensible guides are known. For example:
US2009/0104811 “Extension cord featuring length adjustable ends”, U.S. Pat. No. 8,563,860 “Large loop retractile cord” U.S. Pat. No. 2,206,703 “Wire reinforced cord set” and US2011/0195599 “Flat restorable cable” describe a resiliently axially extensible cable in the form of a helix; and
US2018/0208444 “Hoisting device” describes a resiliently axially extensible current supply cable in the form of a spiral.

A drawback of the cords and cables detailed above is that the outer sleeve is tubular, with the wire extending axially there through. Either the wire must be threaded axially along the length of the outer sleeve, or the outer sleeve (with integrated wire) must be formed (e.g. by extrusion or heat treatment) in the shape of a helix.

EP3123903 “Cable guide for convertible checkout system”, NL8900844, WO2013/156605 “Cable drag chain and modular component system for vertical displacement paths” and US2017/0030485 “Cable guide” describe a resiliently axially extensible guide in the form of a spiral to which a cable is secured. EP3123903 further mentions that an axial end of the guide may be deformed radially outwards as the guide is axially extended, such that the guide (with cable secured thereto) forms a helix with a constant radius of curvature when in an extended condition. A drawback is that when the guide is in a retracted condition, the radius of curvature varies along the length of the guide. Should the wire be substituted with a tube, the tube may become prone to kink in the region of the guide with the smallest radius of curvature.

It is an object of the present invention to provide an axially extensible guide with means for securing a tube thereto, and wherein the guide presents a substantially constant radius of curvature along its length, whether in a neutral condition, an extended condition, or a retracted condition.

### SUMMARY OF THE INVENTION

According to a preferred embodiment of the invention, there is provided an extensible guide including:
a body in the shape of a helix with a substantially constant radius of curvature along the length of the body, the body being axially resiliently deformable between:
(i) a neutral condition, in which the body is not subject to axial compressive or tensile forces;
(ii) an extended condition, in which the body is subject to an axial tensile force; and
(iii) a retracted condition, in which the body is subject to an axial compressive force; and
means for securing a tube to the body, thereby to cause the tube to assume the helical shape of the body,

characterised in that when the body is in the neutral condition, the extended condition or the retracted condition, the radius of curvature of the body remains substantially constant along the length of the body.

Typically, the body is sufficiently elastically deformable such that, when the body subject to an axial tensile force of at least 0.2N, the axial length of the body in the extended condition is at least 1.2 times the axial length of the body in the neutral condition.

Generally, the securing means comprises at least one resilient formation defined by the body, in use to: deform; permit a tube to pass by the resilient formation; and clip the tube to the body.

Preferably, the body is generally U-shaped in axial cross-section, defining: a base; and a pair of arms extending laterally from axial ends of the base.

Typically, the arms converge towards their free ends, thereby providing the resilient formation.

The body is preferably made of a transparent material.

Generally, the body is shaped such that a tube can be radially received by, and secured to the body along: the entire length of the body; or at at least two points per turn of the helical body.

Preferably, the extensible guide further includes a tube received between the body arms, and secured to the body along the entire length of the body by the resilient formation.

Typically, body does not extend circumferentially around the tube, thereby radially exposing a portion of the tube.

Generally, the tube is secured to the body: along the length of the body; or at at least two points per turn of the helical body.

Preferably, the tube is a catheter tube.

Typically, the extensible guide further includes a sleeve that extends around the body with tube secured thereto.

Generally, a first axial end of the sleeve is secured to the tube at or near a first axial end of the body.

Preferably, the tube and sleeve are not secured to each other otherwise than at the first axial end of the sleeve, such that extension of the body from the neutral condition to the extended condition causes relative axial movement of the tube within the sleeve otherwise than at the first axial end of the sleeve.

Typically, the inner diameter of the sleeve is at least 1 mm greater than the outer diameter of the body with tube secured thereto when the helical body is in the neutral condition.

Generally, the axial length of the sleeve is greater than the axial length of the helical portion of the tube when the tube is in the extended condition.

Preferably, the axial length of the sleeve is between 1.2 and 1.5 times the axial length of the body when in the neutral condition.

According to an alternative embodiment, the sleeve is made of an elastomeric material.

Typically, the body with tube secured thereto is snugly received within the sleeve, inducing hoop stresses in the sleeve.

Generally, the sleeve is arranged around the body with tube secured thereto such that extension of the body with tube secured thereto causes corresponding axial extension of the sleeve.

Preferably, the sleeve is arranged around the body with tube secured thereto such that extension of the body from the neutral condition to the extended condition does not cause relative movement between the body with tube secured thereto on the one hand and the sleeve on the other hand.

Typically, the sleeve is made of a transparent material.

Generally, the tube is a co-axial catheter tube.

### BRIEF DESCRIPTION OF THE DRAWINGS

The invention will now be described in more detail, by way of example only, with reference to the accompanying drawings in which:

FIG. 1 is a side view of an extensible guide according to a preferred embodiment of the invention;

FIG. 2 is a perspective view of the extensible guide in FIG. 1;

FIG. 3 is a perspective view of a medical device (in the form of a urethral catheter) with the catheter tube secured to the extensible guide in FIG. 1, and with the extensible guide body in a neutral condition, according to a preferred embodiment of the invention;

FIG. 4 is a side view of the medical device with extensible guide in FIG. 3, with the extensible guide body in an extended condition;

FIG. 5 is an exploded view of the medical device with extensible guide in FIG. 3;

FIG. 6 is a perspective view of a medical device (in the form of a urethral catheter) with the catheter tube secured to the extensible guide in FIG. 1, and with the extensible guide body in a neutral condition, according to an alternative embodiment of the invention;

FIG. 7 is a side view of the medical device with extensible guide in FIG. 6, with the extensible guide body in an extended condition; and

FIG. 8 is an exploded view of the medical device with extensible guide in FIG. 6.

### DESCRIPTION OF THE INVENTION

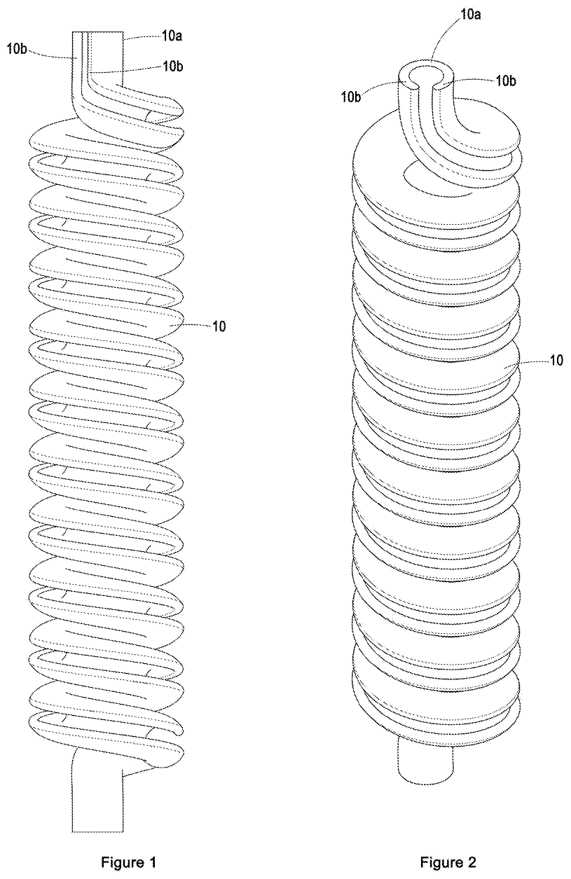

With reference to FIGS. 1 and 2 of the drawings, an extensible guide 10 comprises a body and means for securing a tube 12 to the body.

The body 10 is in the shape of a helix with a substantially constant radius of curvature along the length of the body. The phrase “substantially constant radius of curvature along the length of the body” is intended to mean that the radius of curvature along the length of the body does not vary by more than 5%. The body 10 is made of a transparent elastic material and is axially resiliently deformable between:
(i) a neutral condition, in which the body 10 is not subject to axial compressive or tensile forces;
(ii) an extended condition, in which the body 10 is subject to an axial tensile force; and
(iii) a retracted condition, in which the body 10 is subject to an axial compressive force.

When the body 10 is in any one of the neutral, extended or retracted conditions, the radius of curvature is substantially constant along the length of the helical body 10. However, the radius of curvature along the length of the helical body 10 may vary as the body 10 is deformed between the retracted, neutral and extended conditions. In other words, the radius of curvature along the length of the helical body 10 when in the retracted condition may be greater than the radius of curvature along the length of the helical body 10 when in the extended condition.

In this specification, “axial” is intended to refer to the longitudinal axis of the helical body 10.

The body 10 defines at least 4 helical turns and is sufficiently elastically deformable such that, when the body 10 is subject to an axial tensile force of at least 0.2N (Newtons), the axial length of the body 10 in the extended condition is at least 1.2 times the axial length of the body 10 in the neutral condition. Typically, the body 10 is required to extend between 3 cm and 8 cm (preferably, 5 cm) from the neutral condition to the extended condition. Furthermore, when in the neutral condition, the body 10 is not fully compressed, i.e. adjacent coils of the helical body 10 are not in contact with each other. This permits a degree of axial compression of the body 10 from the neutral condition to the retracted condition, thereby to inhibit transmission of a compressive force along the helical body 10.

The body 10 is generally U-shaped in axial cross-section, defining a base 10a and a pair of arms 10b that extend laterally from the axial ends of the base. The free ends of the arms 10b converge towards each other. Each arm 10b is resilient to enable the arms resiliently to be deformed from a converging configuration to a parallel (or even diverging) configuration, in use to: receive a tube 12 there between; and clip the tube 12 to the body 10 (i.e. between the base 10a and the arms 10b). The arms 10b comprise the “resilient formations” that make up the securing means.

The means for securing a tube 12 to the body 10 is shown as being integral with the body. However, it will be appreciated that the securing means could alternatively comprise loops that extend circumferentially around the body 10. Even further alternatively, instead of each arm 10b extending along the entire length of the base 10a, each arm 10b could define a series of arms that are spaced from each other along the length of the base 10a (i.e. wherein each arm 10b provides a series of “resilient formations”). Importantly the spacing of the arms/“resilient formation” in a series should not be greater than two arms/“resilient formations” (in the series) per turn of the helical body 10.

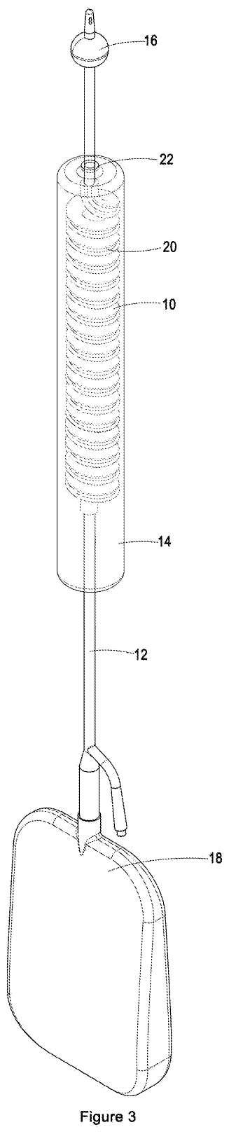

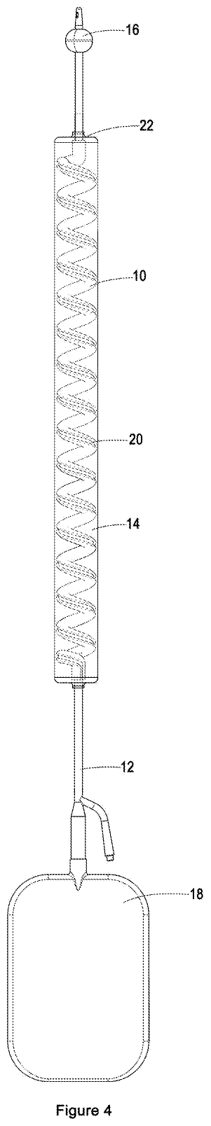

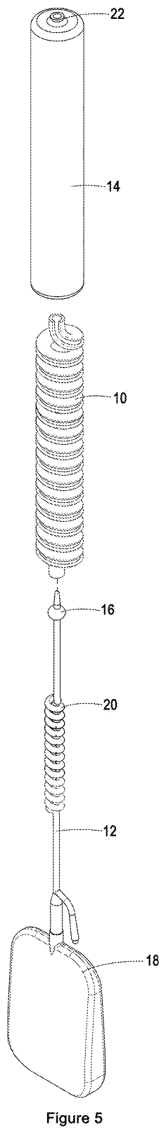

Turning to FIGS. 3 to 5, a tube 12 in the form of a catheter tube can radially be received between the body 10 arms 10b, and thereby be clipped to the body 10. The tube 12 is made of a flexible, transparent silicone (or other polymeric material). By securing the tube 12 to the entire length of the body 10, the body 10 causes the tube 12 to assume the helical shape of the body 10. Accordingly, it is not required that the tube be heat treated/formed/extruded in the shape of a helix. “Radially receiving” the tube 12 should be contrasted against “axially receiving” the tube 12, which latter manner would require the tube 12 to be pushed axially along the entire length of the body 10.

It will be appreciated that, since the helical body 10 deforms the tube 12 into a helix with a substantially constant radius of curvature along the axial length of the tube 12, the risk of the tube 12 kinking (and the consequent risk of blocking fluid flow along the tube 12) is reduced. It will also be appreciated that, to force the tube 12 to adopt the helical shape of the body 10, the tube 12 could alternatively be secured to the body 10 at at least two points per turn of the helical body 10, instead of being secured to the body 10 along the entire length of the body 10.

The body 10 does not extend circumferentially around the tube 12. Instead, the free ends of the arms 10b are spaced from each other, exposing a radial portion of the tube 12. This facilitates visual inspection of the tube 12 and fluid flow along the tube 12.

The tube 12 includes an inflatable bladder 16 at a first axial end, and a bag 18 at a second axial end. It will be appreciated that, although FIGS. 3 to 5 show a medical device in the form of a urethral catheter, it could alternatively be an intravenous drip or infusion device. Furthermore, this invention is not limited to medical devices.

In use: the inflatable bladder 16 is secured at or near the outlet of a patient's bladder; and the bag 18 is attached to the patient's upper leg. Should the patient splay his legs (e.g. by moving one leg to which the bag 18 is attached out of a vehicle), the resilient body 10 (with tube 12) extends and retracts in sympathy with relative displacement of the inflatable bladder 16 and bag 18 (or attachment of the tube 12 to the thigh of the patient), thereby reducing the tensile and compressive forces transmitted by the tube 12 upon the inflatable bladder 16.

Should the patient's leg to which the bag 18 is attached be covered by a bed sheet or trouser leg, it will be appreciated that extension or retraction of the helical body 10 (with tube 12) may become snagged on the sheet or trouser and tug on the inflatable bladder 16. To inhibit such snagging, a sleeve 14 extends around the axial length of the helical body 10 (with tube 12), covering the helical portion 20 of the body 10 (with tube 12).

The sleeve 14 is made of a flexible material. Preferably, the sleeve 14 is transparent, in use, to permit inspection through the sleeve 14 of fluid flow along the helical formed portion of the tube 12 (in the region of the body 10).

A first axial end 22 of the sleeve 14 is secured to the tube 12 at or near a first axial end of the body 10 (i.e. spaced along the tube 12 not more than 50 mm from the first axial end of the body 10, in a direction away from the second axial end of the body 10). The sleeve 14 is not secured to either the body 10 or the tube 12 otherwise than at the first axial end of the sleeve 14. Furthermore, the inner diameter of the sleeve 14 is at least 1 mm greater than the outer diameter of the helical body 10 (with tube 12) in the neutral condition. This facilitates free relative axial movement between the sleeve 14 and the helical body 10 (with tube 12) upon extension of the helical body 10 (with tube 12) from the neutral condition to the extended condition. To ensure that the sleeve 14 fully covers the helical body 10 (with tube 12) in the extended condition: the axial length of the sleeve 14 is greater than the axial length of the body 10 when in the extended condition; more specifically, the axial length of the sleeve 14 is between 1.2 and 1.5 times the axial length of the body 10 in the neutral condition.

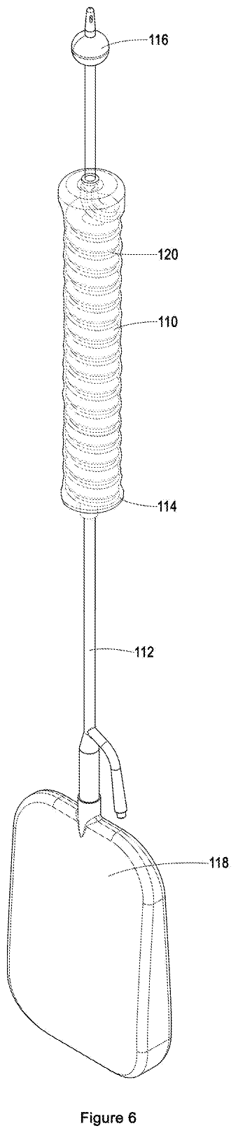

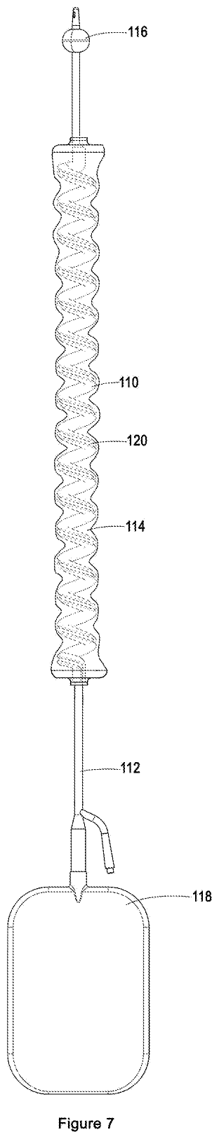

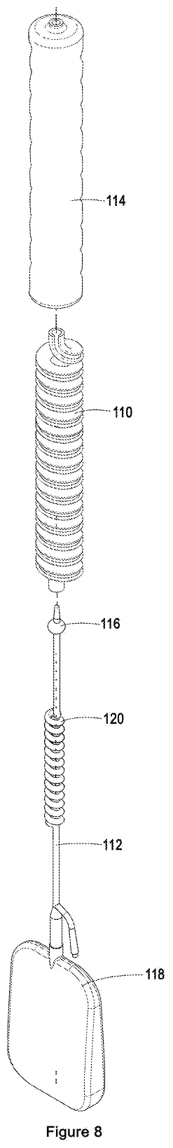

FIGS. 6 to 8 of the drawings show an alternative arrangement for the sleeve 114. The sleeve 114 is made of a transparent, elastomeric material and stretched over the helical body 110 (with tube 112), such that the helical body 110 (with tube 112) is snugly received within the sleeve 114, inducing hoop stresses in the sleeve 114. In such arrangement, friction between the helical body 110 (with tube 112) and the sleeve 114:
causes axial extension of the sleeve 114 in sympathy with axial extension of the helical body 110 (with tube 112) from the neutral condition shown in FIG. 6 to the extended condition shown in FIG. 7; and
inhibits relative movement between the helical body 110 (with tube 112) and the sleeve 112 upon extension of the helical body 110 (with tube 112) from the neutral condition to the extended condition.

It will be appreciated that by covering the helical body 10, 110 (with tube 12, 112), the sleeve 14, 114 inhibits snagging of the helical body 10, 110 (with tube 12, 112) on adjacent objects. It will be even further appreciated that, with:
(i) the helical body 10, 110 forming the tube 12, 112 into a helix with a substantially constant radius of curvature along the length of the helix; and
(ii) the sleeve 14, 114 covering and protecting the helical body 10, 110 (with tube 12, 112),

kinking of the tube 12, 112 is inhibited.

1. An extensible guide including:
a body in the shape of a helix with a substantially constant radius of curvature along the length of the body, the body being axially resiliently deformable between:
(i) a neutral condition, in which the body is not subject to axial compressive or tensile forces;
(ii) an extended condition, in which the body is subject to an axial tensile force; and
(iii) a retracted condition, in which the body is subject to an axial compressive force; and
means for securing a tube to the body, thereby to cause the tube to assume the helical shape of the body,
characterised in that when the body is in the neutral condition, the extended condition or the retracted condition, the radius of curvature of the body remains substantially constant along the length of the body.

2. The extensible guide according to claim 1, wherein the body is sufficiently elastically deformable such that, when the body is subject to an axial tensile force of at least 0.2N, the axial length of the body in the extended condition is at least 1.2 times the axial length of the body in the neutral condition.

3. The extensible guide according to claim 2, wherein the securing means comprises at least one resilient formation defined by the body, in use to: deform; permit a tube to pass by the resilient formation; and clip the tube to the body.

4. The extensible guide according to claim 3, wherein the body is generally U-shaped in axial cross-section, defining: a base; and a pair of arms extending laterally from axial ends of the base.

5. The extensible guide according to claim 4, wherein the arms converge towards their free ends, thereby providing the resilient formation.

6. The extensible guide according to claim 5, wherein the body is shaped such that a tube can be radially received by, and secured to the body along: the entire length of the body; or at at least two points per turn of the helical body.

7. The extensible guide according to claim 6, wherein the body is made of a transparent material.

8. The extensible guide according to claim 7, further including a tube received between the body arms, and secured to the body along the entire length of the body by the resilient formation.

9. The extensible guide according to claim 8, wherein the body does not extend circumferentially around the tube, thereby radially exposing a portion of the tube.

10. The extensible guide according to claim 9, wherein the tube is secured to the body: along the length of the body; or at at least two points per turn of the helical body.

11-23. (canceled)
